(19) United States
(12) Patent Application Publication
(10) Pub. No.: US 2009/0299082 A1
(43) Pub. Date: Dec. 3, 2009

(54) EPOXY FUNCTIONALIZED CARBON NANOTUBES AND METHODS OF FORMING THE SAME

(75) Inventors: Charles Baudot, Singapore (SG); Maria Viviana Volpe, Pozzuoli (IT); Jeng Chien Kong, Singapore (SG); Cher Ming Tan, Singapore (SG)

Correspondence Address: STMICROELECTRONICS, INC., MAIL STATION 2346, 1310 ELECTRONICS DRIVE, CARROLLTON, TX, 75006, US

(73) Assignees: STMicroelectronics Asia Pacific Pte Ltd., Singapore (SG); STMicroelectronics Srl, Agrate Brianza (IT); Nanyang Technological University, Singapore (SG)

(21) Appl. No.: 12/410,630

(22) Filed: Mar. 25, 2009

(30) Foreign Application Priority Data
Mar. 25, 2008 (IT) TO2008A000225

(51) Int. Cl. C07D 303/04

(52) U.S. Cl. 549/513; 549/512; 977/752

(57) ABSTRACT

The invention relates to epoxy functionalized carbon nanotubes (CNTs) and methods of forming the same, and more particularly to inclusion of the epoxy functionalized CNTs as fillers in electronic applications, e.g., semiconductor devices and device packaging. More particularly, CNT-based epoxy resin composites are employed as materials for electronic packaging applications and the inclusion of CNTs as fillers chemically linked to epoxy resin macromolecules. The resulting materials showed improved chemical-physical features in terms of mechanical, thermal and electrical properties.

This application claims the benefit of Italian Patent Application No. TO2008A000225, filed on Mar. 25, 2008, which is hereby incorporated by reference for all purposes as if fully set forth herein.

### BACKGROUND OF THE INVENTION

1. Field of the Invention

The invention relates to epoxy functionalized carbon nanotubes (CNTs) and methods of forming the same, and more particularly to inclusion of the epoxy functionalized CNTs as fillers in composite materials for electronic applications, e.g., semiconductor devices and device packaging.

2. Discussion of the Related Art

Epoxy and polyepoxide resins are composed of a thermosetting polymer that cures (polymerizes and crosslinks) when mixed with a catalyzing agent or “hardener”. Several common epoxy resins result from the condensation of two reactants: epichlorohydrin and bisphenol-A. The details of the chemical mechanism of epoxidic resin preparation from bisphenol-A and epichlorohydrin condensation are shown FIG. 1.

Generally, epoxy resins are known for their excellent adhesion properties; chemical, thermal and mechanical resistances; and electrical insulating properties. Thus the application fields range from adhesives to composite materials for high resistance mechanical applications. The pure epoxy resin average chemical-physical properties are reported in Table 1 as follows:

TABLE 1

| | | Coefficient of | | |
|---|---|---|---|---|
| | Thermal | Thermal | Young's | Dielectric |
| Conductivity | conductivity | Expansion(CTE) | Modulus | constant |
| None | 0.1 W/mK | 310 ppm/° C. | 6200 MPa | 3.1 |

Properties of epoxy resins may be modified by filling the polymeric matrices with inorganic particles. Inorganic particles may be added to the resins, e.g., metals may be incorporated in the resins to switch the resins from an electrically insulating material to a conductive material. Also, the resins may be filled with organic materials, e.g., carbon fibers, to enhance the mechanical resistance.

In the field of semiconductors, manufacturing epoxy resins are employed as materials for the packaging of electronic devices. More particularly, electronic packaging is often an important step in the process as it deals with the circuit element's interconnections, the device cooling, the total device protection and the device's mechanical support.

### SUMMARY OF THE INVENTION

The invention is directed to epoxy functionalized carbon nanotubes (CNTs) and methods of forming the same that substantially obviate one or more of the problems due to limitations and disadvantages of the related art.

An advantage of the invention is to provide epoxy functionalized CNTs with improved properties.

Another advantage of the invention is using epoxy functionalized CNTs in electrical applications, e.g., semiconductor devices and device packaging.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the invention, an embodiment of the invention is directed towards a method of forming epoxy functionalized carbon nanotubes (CNTs). The method includes functionalizing carbon nanotubes by reacting with organolithium reactants followed by a nucleophilic substitution to form the epoxy functionalized carbon nanotubes.

Another embodiment of the invention is directed towards a packaging of electronic components including epoxy functionalized carbon nanotubes. The epoxy functionalized carbon nanotubes are made by functionalizing the carbon nanotubes by reacting with organolithium reactants followed by a nucleophilic substitution to form the epoxy functionalized carbon nanotubes.

Yet another embodiment of the invention is directed towards an epoxy functionalized carbon nanotube. The epoxy functionalized carbon nanotubes are made by functionalizing the carbon nanotubes by reacting with organolithium reactants and nucleophilic substitution to form the epoxy functionalized carbon nanotubes.

Still another embodiment of the invention is directed towards a method of forming epoxy functionalized carbon nanotubes (CNTs). The method includes functionalizing carbon nanotubes with a Prato reaction nucleophilic substitution to form the epoxy functionalized carbon nanotubes.

Another embodiment of the invention is directed towards a packaging of electronic components including epoxy functionalized carbon nanotubes. The epoxy functionalized carbon nanotubes are made by functionalizing carbon nanotubes with a Prato reaction nucleophilic substitution to form the epoxy functionalized carbon nanotubes.

Yet another embodiment of the invention is directed towards an epoxy composite containing functionalized carbon nanotubes. The epoxy functionalized carbon nanotubes are made by functionalizing carbon nanotubes with a Prato reaction nucleophilic substitution to form the epoxy functionalized carbon nanotubes.

Still another embodiment of the invention is directed towards a method of forming epoxy functionalized carbon nanotubes (CNTs). The method of forming epoxy functionalized carbon nanotubes (CNTs) includes functionalizing carbon nanotubes with a Bingel reaction and transesterification of ethoxylic groups of the functionalized carbon nanotubes by reacting with glycidol molecules to form the epoxy functionalized carbon nanotubes.

Another embodiment of the invention is directed towards a packaging of electronic components including epoxy functionalized carbon nanotubes. The epoxy functionalized carbon nanotubes are made by functionalizing carbon nanotubes with a Bingel reaction and transesterification of ethoxylic groups of the functionalized carbon nanotubes by reacting with glycidol molecules to form the epoxy functionalized carbon nanotubes.

Yet another embodiment of the invention is directed towards an epoxy composite containing functionalized carbon nanotubes. The epoxy functionalized carbon nanotubes are made by functionalizing carbon nanotubes with a Bingel reaction and transesterification of ethoxylic groups of the functionalized carbon nanotubes by reacting with glycidol molecules to form the epoxy functionalized carbon nanotubes.

Still another embodiment of the invention is directed towards a method of forming epoxy functionalized carbon nanotubes (CNTs). The method of forming epoxy functionalized carbon nanotubes (CNTs) includes functionalizing carbon nanotubes with a Prato reaction and epoxidation reaction to form the epoxy functionalized carbon nanotubes.

Another embodiment of the invention is directed towards a packaging of electronic components including epoxy functionalized carbon nanotubes. The epoxy functionalized carbon nanotubes are made by functionalizing carbon nanotubes with a Prato reaction and epoxidation reaction to form the epoxy functionalized carbon nanotubes.

Yet another embodiment of the invention is directed towards an epoxy composite containing functionalized carbon nanotubes. The epoxy functionalized carbon nanotubes are made by functionalizing carbon nanotubes with a Prato reaction and epoxidation reaction to form the epoxy functionalized carbon nanotubes.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 illustrates a related art chemical mechanism for epoxidic resin preparation from bisphenol-A and epichlorohydrin condensation;

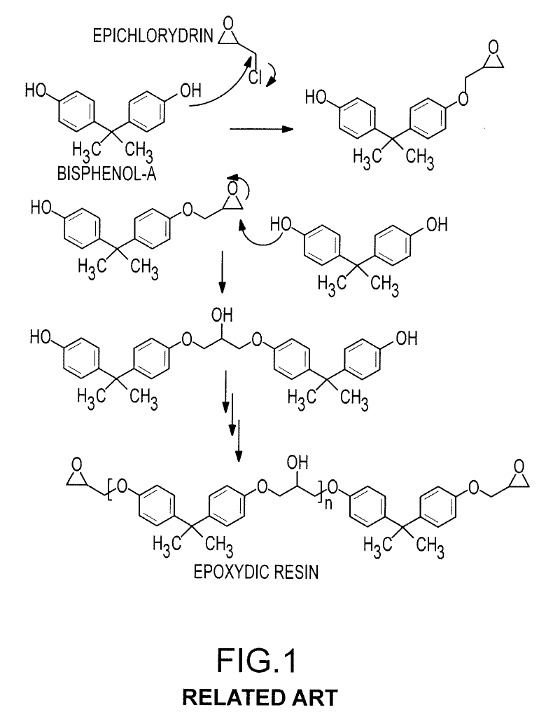

FIG. 2 illustrates Young's modulus of functionalized multiwalled carbon nanotubes based nanocomposite resins;

FIG. 3 illustrates step 1 of Strategy A—liquid-phase CNTs chemical functionalization by butyllithium reactant in toluene medium;

FIG. 4 illustrates step 2 of Strategy A—nucleophilic attack from carbanions on CNTs sidewall to epichloridrin molecules and resulting epoxy groups attaching onto CNTs;

FIG. 5 illustrates a flowchart of Strategy A according to Example 1;

FIG. 6 illustrates a Fourier Transform infrared (FTIR) absorption spectra of Example 1;

FIG. 7 illustrates a Prato reaction mechanism according to Strategy B;

FIG. 8 illustrates a nucleophilic attack from pyrrolidine nitrogen atom to epichlorohydrin molecules and functionalization of CNTs by terminal epoxy groups under ambient conditions according to Strategy B;

FIG. 9 illustrates Example 2 according to step 1 of Strategy B;

FIG. 10 illustrates a chemical mechanism of CNTs sidewall functionalization by the Bingel reaction according to Strategy C;

FIG. 11 illustrates a transesterification mechanism of ethoxylic groups replacement by glycidol molecules (nucleophilic substitution reaction) mechanism according to Strategy C;

FIG. 12 illustrates Example 3 according to step 2 of Strategy C;

FIG. 13 illustrates a Fourier Transform infrared (FTIR) absorption spectra measured on Example 3;

FIG. 14 illustrates a Prato reaction mechanism with N-acetylglycine and para formaldehyde according to Strategy D; and

FIG. 15 illustrates a Corey-Chaykovsky epoxydation reaction mechanism according to Strategy D.

### DETAILED DESCRIPTION OF THE ILLUSTRATED EMBODIMENTS

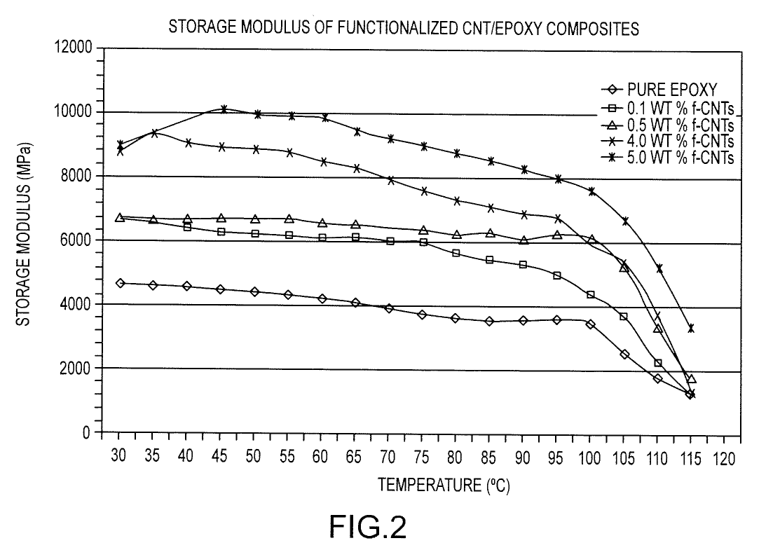

Embodiments of the invention are directed towards epoxy functionalized CNTs and methods of forming the same. The epoxy functionalized CNTs have improved thermal and mechanical properties over the related art. Epoxydic composite materials based on the use of functionalized CNTs as nanofillers of polymeric matrices are prepared using different weight percentages of created (functionalized) multiwalled carbon nanotubes (MWCNTs) and show improvements in physical features; these improvements include high mechanical resistance and thermal and electronic conductivities. The thermo-mechanical behavior was examined by measuring the improvement in Young modulus vs. temperature for different MWCNTs contents in the composite samples. The results are reported in FIG. 2.

Referring to FIG. 2, it is shown that the change of Young's modulus of MWCNTs-based composite resins is a function of different weight percentages. The mechanical resistance enhances linearly as the weight percentage of MWCNTs increases. Also, to obtain composites having further chemical-physical improvements, high homogeneous CNTs dispersion is desired. This condition generally represents a process issue due to CNTs low solubility and the trend to create aggregates and phase separation.

Embodiments of the invention are directed towards epoxy functionalized CNTs and methods of forming the same. For example, methods of forming epoxy resin composites including highly dispersed CNTs as fillers, where the high dispersion is due to the chemical linkage between functionalized CNTs and the macromolecules of the epoxy resin matrix. The procedures and methods in embodiments of the invention incorporate both MWCNTs and single walled nanotubes (SWNTs). Also, the epoxy resin nanocomposites may be used in a variety of electrical devices, e.g., semiconductor devices and packages.

In embodiments of the invention, MWCNT and SWNT may be obtained from a variety of sources as known in the art. In preferred embodiments the MWCNTs were obtained from Shanzhen Nanotechnology Co. Ltd of China and a have a claimed purity greater than about 95%, a diameter in the range from about 10 nm to about 20 nm, a length in the range from about 5 μm to about 15 μm, and an amorphous carbon content of less than about 3%. In other preferred embodiments the SWNTs were also obtained from Shanzhen Nanotechnology Co. Ltd of China and have a purity greater than about 90%, a content of SWNTs greater than about 60%, a diameter of less than about 2 nm, a length of less than about 20 μm, and amorphous carbon content of less than about 5%. Commercial available epoxy resins were used in a preferred embodiment. The commercial available epoxy resins were obtained from Wee Tee Tong Chemicals Pte Ltd, Singapore, having a model number of EPICOTE 1006-A system, an appearance of clear liquid, a flashpoint of about 100° C., a specific gravity of about 1.10, low viscosity, a gel time of about 67 mins, and a pot life of about 47 mins.

In embodiments of the invention functionalized CNTs epoxy composites were formed with a two step procedure: the first step is directed towards chemical functionalization of CNTs with epoxy terminal groups, and the second step is directed towards mixing the epoxy-functionalized CNTs with epoxy resins and hardeners. More particularly, four different methods (Strategies A-D) for forming epoxy functionalized carbon nanotubes (CNTs) will now be described.

Strategy A is directed towards a two part process: step 1, CNTs chemical functionalization by organolithium reactants, and step 2, nucleophilic substitution with halogeno or hydroxyl oxacylcopropanes, e.g., epichlorohydrin, glycidol, and the like. Turning now to Strategy A.

### Strategy A:

Step 1. CNTs Chemical Functionalization by Organolithium Reactants; and

Step 2. Nucleophilic Substitution with Epichlorohydrin.

This chemical functionalization strategy consists of a ‘one-pot’ procedure, e.g., batch reaction, where the two reactive steps are carried out in the same chemical environment without a sample work-up between step 1 and step 2.

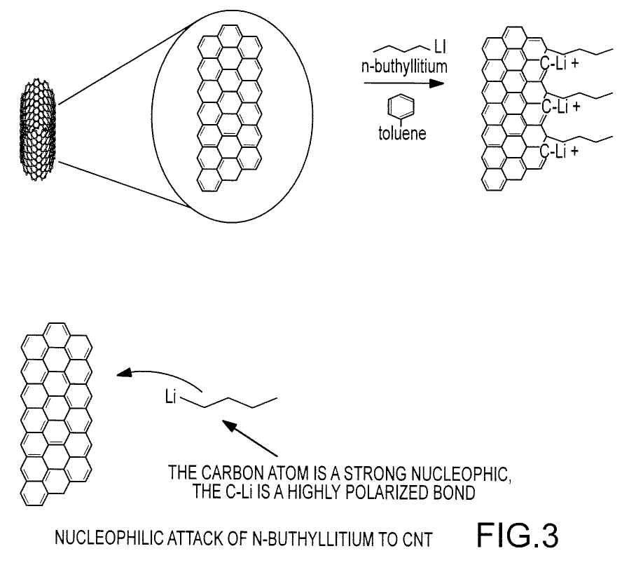

Step 1 of Strategy A is directed towards CNTs functionalization by organolithium reactants. The functionalization of CNTs is carried out recurring to a liquid phase procedure, employing toluene as solvent. In a preferred embodiment, this functionalization is carried out with n-butyllithium as reactant, e.g., 2.0 M n-Butyllithium solution in cyclohexane and Toluene ReagentPlus® supplied by Sigma-Aldrich. The functionalization according to embodiments of the invention is described by Blake, et al., A Generic Organometallic Approach toward Ultra-Strong Carbon Nanotube Polymer Composites, J. Am. Chem. Soc. (2004), 126, pp. 10226-10227; and Viswanathan, et al., Single-Step in Situ Synthesis of Polymer-Grafted Single-Wall Nanotube Composites, J. Am. Chem. Soc. (2003), 125, pp. 9258-9259, all of which are hereby incorporated by reference as if fully set forth herein. The colloidal mixture is maintained under inert atmosphere during the reaction process as the n-butyllithium tends to react with atmospheric humidity. The CNTs functionalization chemical mechanism consists in the nucleophilic attack of n-butyllithium molecules to CNTs carbon atoms resulting in the electronic shifts on the neighboring carbon atoms and formation of intermediate ionic species. FIG. 3 illustrates step 1 of Strategy A, liquid-phase CNTs chemical functionalization by butyllithium reactant in toluene medium.

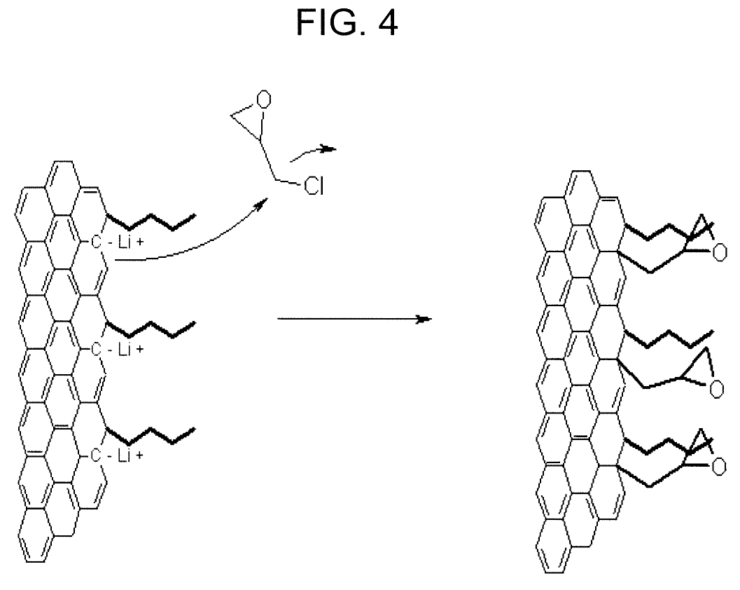

Step 2 is directed towards a nucleophilic substitution with epichlorohydrin. This step involves adding epichlorohydrin to the reaction mixture, e.g., epichlorohydrin 99%, supplied by Sigma Aldrich. Butyl-functionalized CNTs are almost a nucleophilic agent because of carbanions on the sidewall, so adding epichlorohydrin results in the nucleophilic attack from the CNTs sidewall carbanions to epichlorohydrin molecules at the carbon atom bearing chlorine group. The chemical mechanism of this reaction is shown in FIG. 4 and illustrates step 2 of Strategy A, a nucleophilic attack from carbanions on CNTs sidewall to epichloridrin molecules and resulting epoxy groups attaching onto CNTs.

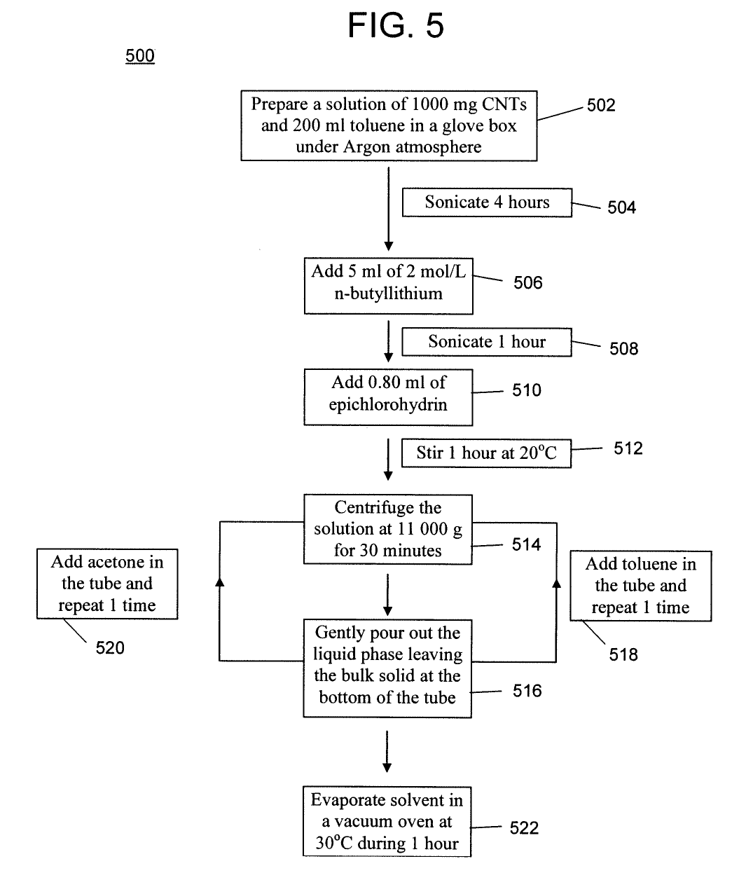

Example 1 is a method of forming epoxy functionalized CNTs according to Strategy A. FIG. 5 is a flowchart of Example 1. Referring to FIG. 5, the flowchart is generally depicted as reference 500. In step 502, a solution was prepared of 100 mg of CNTs and 200 ml of toluene in a glove box under an argon atmosphere. In step 504, the solution was sonicated for about 4 hours. In step 506, 5 ml of 2 mol/L of n-butyllithium was added. In step 508, this solution was sonicated for about 1 hour. In step 510, about 0.80 ml of epichlorohydrin was added. In step 512, the mixture was stirred for about an hour at 20° C. In step 514, the solution was centrifuged at 11,000 g for about 30 minutes. In step 516, the liquid phase was gently removed by pouring out the liquid phase and leaving the bulk solid at the bottom of the container. In step 518, toluene was added and steps 514 and 516 were repeated. In step 520, acetone was added and steps 514 and 516 were repeated. In step 522, evaporation of the solvent in a vacuum oven at 30° C. for about 1 hour was conducted, thereby forming an epoxy functionalized CNT powder.

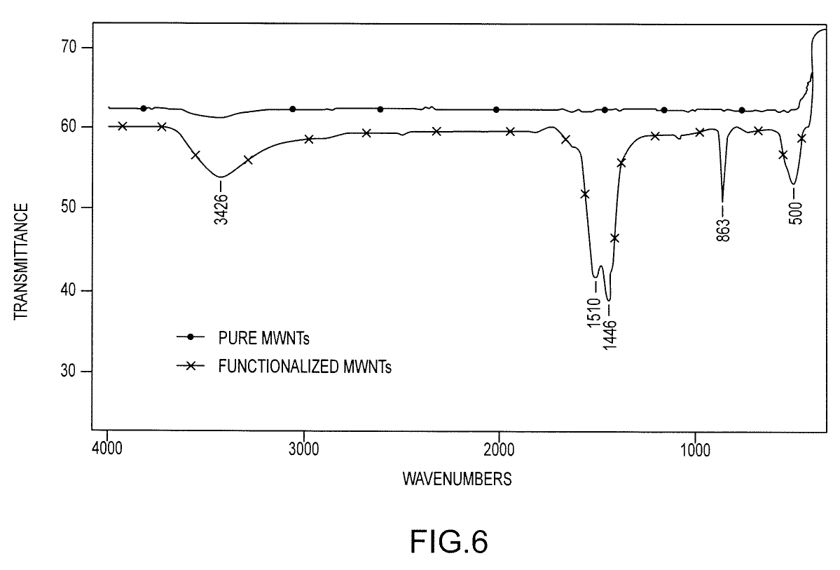

FIG. 6 illustrates Fourier Transform infrared (FTIR) absorption spectra of Example 1. The FTIR absorption spectra is also reported in Table 2. These results confirm epoxy functionalized MWCNTs. In particular, it is shown that epoxy groups are present due to the absorption in a range from about 805 to about 880.

TABLE 2
FTIR absorbtion data of epoxy-functionalized MWCNTs

| INDEX | RANGE(cm⁻¹) | FRAGEMENT | COMMENT |
|---|---|---|---|
| a | 485-540 | Straight chain alkanes | C—C skeleton |
| | | | vibration |
| b | 805-880 | Monosubstituted epoxide | Ring vibration |
| c | 1400-1415 | Monosubstituted epoxide | CH₂ deformation |
| | 1475-1500 | | vibration |
| d | 3400-3590 | Intermolecular hydrogen | Water absorption |
| | | bond | |

Strategy B is directed towards a two part process for forming epoxy functionalized CNTs. The process includes two steps: step 1, functionalization of CNTs by a Prato reaction, and step 2, nucleophilic substitution with halogeno or hydroxyl oxacylcopropanes, e.g., epichlorohydrin, glycidol, and the like. Turning now to Strategy B.

### Strategy B:

Step 1. CNTs Functionalization by the Prato Reaction; and

Step 2. Nucleophilic Substitution with Epichlorohydrin.

This functionalization strategy also consists of two steps. In embodiments of the invention, the first step includes a Prato reaction, based on a 1,3-dipolar cycloaddition of azomethine ylides on the nanotube. The Prato reaction is described by Prato, et al., Synthesis and applications of fulleropyrrolidines, Synthetic Metals (1996), 77, pp. 89-91; Vasillios, et al., Organic Functionalization of Carbon Nanotubes, J. Am. Chem. Soc. (2002), 124, 5, pp. 760-761; Percec, et al., Transformation of a Spherical Supramolecular Dendrimer into a Pyramidal Columnar Supramolecular Dendrimer Mediated by the Flurophbic Effect, Angew. Chem. (2003), 115, pp. 4338-4341, all of which are hereby incorporated by reference as if fully set forth herein. Subsequently, step 2 includes a substitution reaction of epichlorohydrin occurring on the grafted molecule in order to add the functional group on the latter.

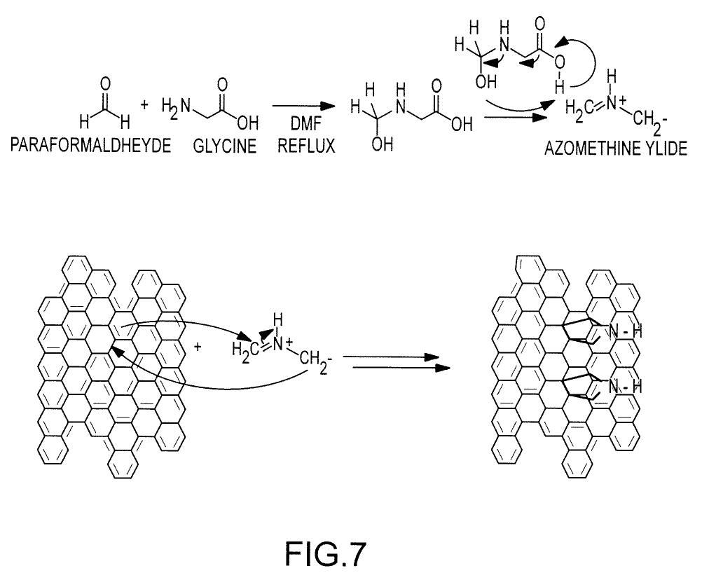

More particularly, in step 1, functionalization of CNTs by the Prato reaction includes reacting an amino acid with an aldehyde to form an azomethine ylide. The procedure which is followed is the one reported in literature by Vasillios, et al., Organic Functionalization of Carbon Nanotubes, J. Am. Chem. Soc. (2002), 124, 5, pp. 760-761. The reaction is carried out under refluxing in dimethylformamide at about 140° C. and using glycine and paraformaldehyde as reactants, e.g., para formaldehyde purum, supplied by Fluka, Glycine ReagentPlus® and dimethylformamide anhydrous, supplied by Sigma Aldrich. The mixture is left to react for about 96 hours. Glycine and paraformialdehycie react together by following the decarboxylation route to form the azomethine ylides. The azomethine ylide is formed and it attacks the CNT sidewalls and tips by 1,3-dipolar cycloaddition as shown in FIG. 7, illustrating a Prato reaction mechanism (cycloaddition mechanism). Then, the functionalized CNTs are filtered out using vacuum filtering, washed using ethanol and dried in a vacuum oven at about 30° C. for about two hours.

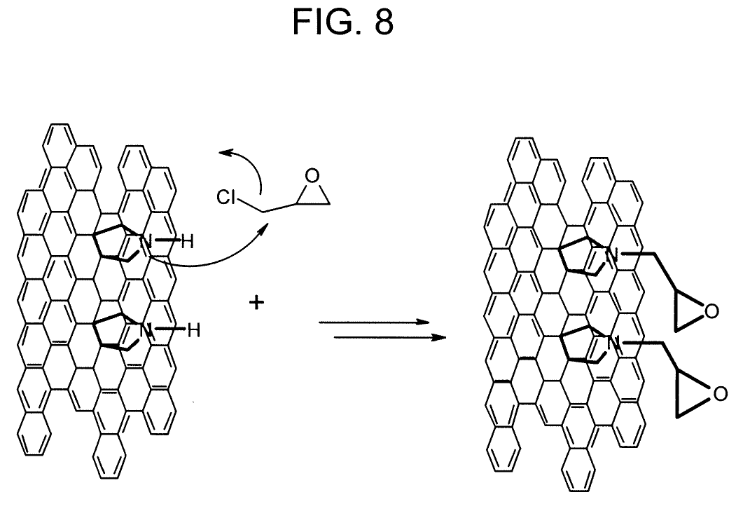

Step 2 of Strategy B is directed towards a nucleophilic substitution reaction using epichlorohydrin. During this step, the CNTs pre-functionalized using the Prato reaction are stirred in epichlorohydrin so that a nucleophilic substitution reaction occurs forming HCl as a byproduct. The mechanism of nucleophilic attack from pyrrolidine nitrogen atom to epichlorohydrin molecules and functionalization of CNTs by terminal epoxy groups of this reaction is shown in FIG. 8. The nucleophilic attack from pyrrolidine nitrogen atom to epichlorohydrin molecules and functionalization of CNTs by terminal epoxy groups is under ambient conditions. The CNTs are then filtered out, washed using acetone and dried in a vacuum oven at about 30° C. during one hour, thereby forming epoxy functionalized carbon nanotubes (CNTs).

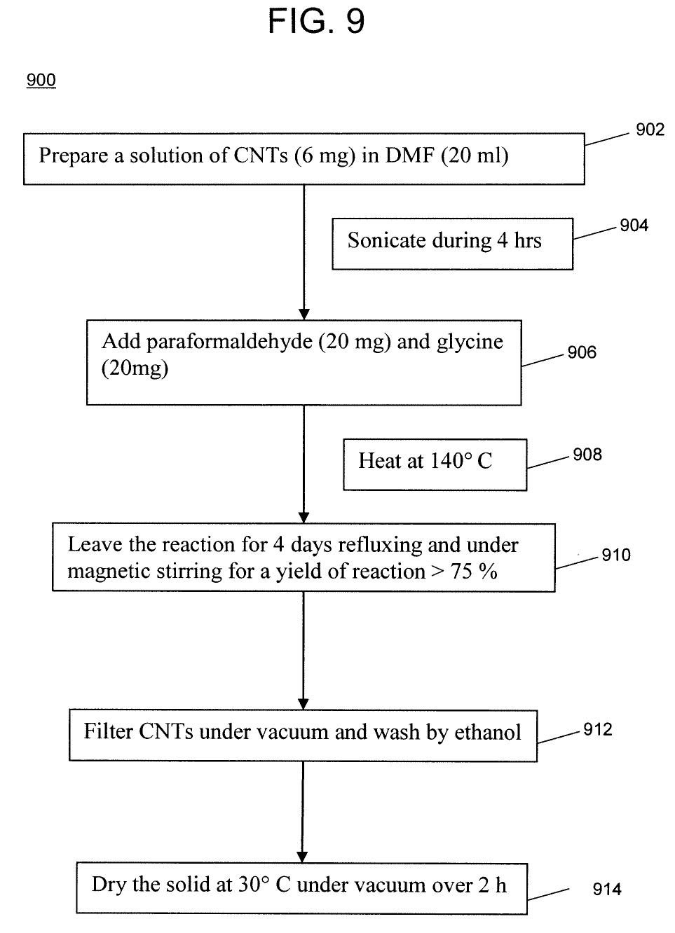

FIG. 9 illustrates Example 2 according to step 1 of Strategy B. Example 2 is shown by flowchart 900 and is directed towards CNT functionalization by the Prato reaction. In step 902, a solution was prepared by mixing 6 mg of CNTs in 20 ml of dimethylformamide (DMF). In step 904, the solution was sonicated for about 4 hours. In step 906, about 20 mg of paraformaldehyde and about 20 mg were added. In step 908, the solution was heated to about 140° C. In step 910, the solution was reacted for about 4 days reluxing and under magnetic stirring for a yield of reaction greater than about 75%. In step 912, the CNTs were filtered under vacuum and ethanol washing. In step 914, the solid was dried at 30° C. under a vacuum over 2 hours, thereby forming functionalized CNTs.

### Strategy C:

This embodiment is directed towards and includes a two step process for forming functionalized CNTs. Step 1 is directed towards a cyclopropanation reaction followed by step 2, a transesterification reaction. The two reactive steps are separated by an intermediate sample work up. Turning now to Strategy C.

Step 1 of this embodiment includes CNTs functionalization by the Bingel reaction. The Bingel reaction is a liquid-phase reaction known to one of ordinary skill in the art for chemical modification of fullerene. The chemistry of fullerene is very close to the chemistry of carbon nanotubes, however the CNTs may show less reactivity and often some experimental parameters, such as reaction time and temperature, may be different than those tuned for fullerene and its derivatives. The Bingel reaction is also known as cyclopropanation reaction since it leads to cyclopropane ring formation on the CNTs sidewall as set forth in Coleman, et al, Functionalization of Single-Walled Carbon Nanotubes via the Bingel Reaction, J. Am. Chem. Soc. (2003), 125, pp. 8722-8723; and Worsely, et al., Long-Range Periodicity in Carbon Nanotube Sidewall Functionalization, Nano Lett., (2004), 4, 8, pp. 1541-1546, all of which are hereby incorporated by reference as if fully setforth herein. The chemical mechanism involves the attack of a nucleophilic carbanion generated in situ at CNTs.

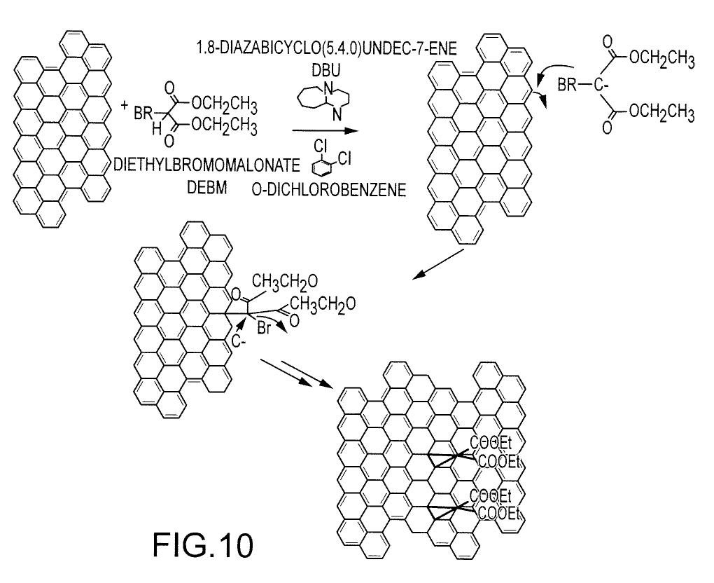

In particular, the main reactant, diethylbromomalonate (DEBM) is activated by the base, 1,8-diazabiciclo [5.4.0]undecene (DBU), by deprotonation and then the nucleophilic addition of the resulting carbonanion occurs on the CNTs. This mechanism is followed by a cyclization rearrangement with the formation of a cyclopropane ring bearing two ethoxycarbonyl groups. The chemical mechanism of CNTs sidewall functionalization by the Bingel reaction is shown in FIG. 10. The procedure followed in the experimental description is reported in literature as set forth in Coleman, et al, Functionalization of Single-Walled Carbon Nanotubes via the Bingel Reaction, J. Am. Chem. Soc. (2003), 125, pp. 8722-8723; and Worsely, et al., Long-Range Periodicity in Carbon Nanotube Sidewall Functionalization, Nano Lett., (2004), 4, 8, pp. 1541-1546.

CNTs are dissolved in o-dichlorobenzene and left for a predetermined time under ultrasonication in order to obtain a well-dispersed colloidal suspension, e.g., for about 1 hour under ultrasonication. Then, DEBM and DBU are added to the mixture and left to react under ultrasonication. In a preferred embodiment, the reaction is for about 20 hours under o-dichlorobenzene ReagentPlus®, 1.8-diazabiciclo [5.4.0]undecene (DBU) and diethylbromomalonate of about 85 to about 95% (DEBM) supplied by Sigma Aldrich. Next, functionalized-CNTs samples are filtered out under a vacuum and washed several times with ethanol anhydrous and dried in a vacuum oven.

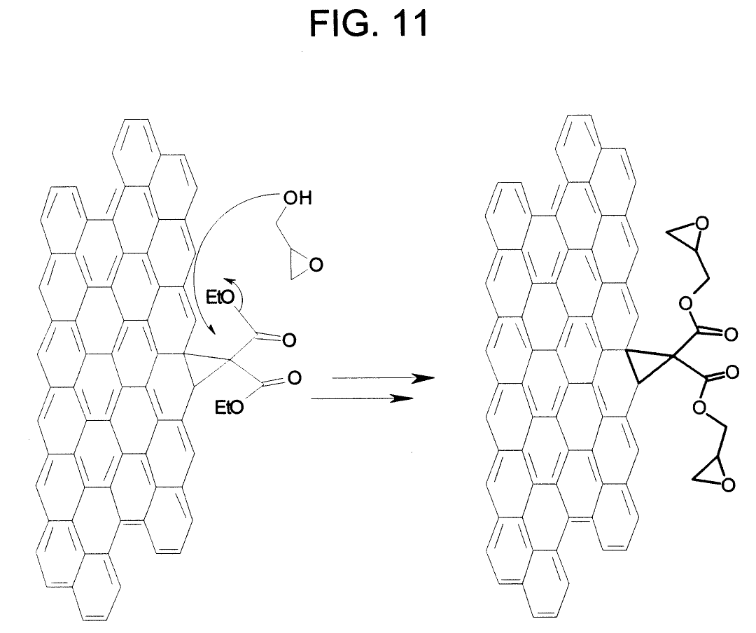

Step 2 of Strategy C is directed towards a transesterification with glycidol. More specifically, this step involves the transesterification of ethoxylic groups by glycidol molecules. The reaction is carried out by dissolving diethoxycarbonyl-functionalized CNTs in liquid glycidol, e.g., glycidol 96% supplied by Sigma Aldrich, and leaving the reaction mixture under magnetic stirring and at room temperature until total conversion. The transesterification mechanism of ethoxylic groups replacement by glycidol molecules (nucleophilic substitution reaction) mechanism is shown in FIG. 11.

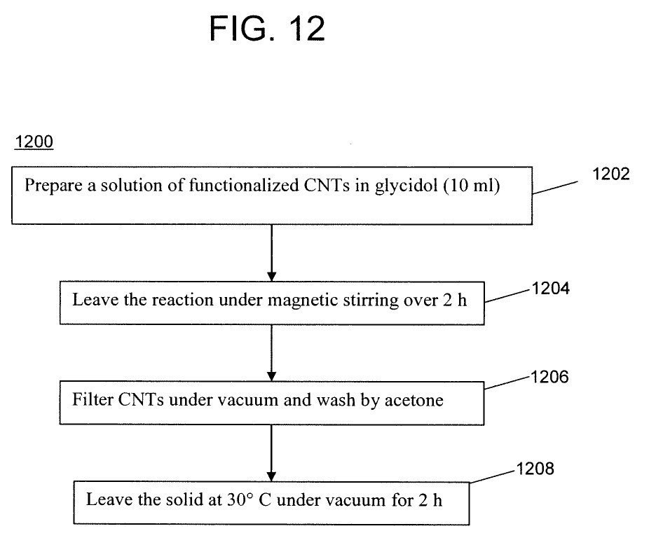

FIG. 12 illustrates Example 3 according to step 2 of Strategy C. Example 3 is shown by flowchart 1200 and is directed towards step 2, a nucleophilic substitution reaction using glycidol, thereby forming epoxy functionalized CNTs. In step 1202, a solution of functionalized CNTs in 10 ml of glycidol was prepared. In step 1204, a reaction under magnetic stirring for about 2 hours and at room temperature was conducted. In step 1206, CNTs were filtered under a vacuum and washed in acetone. In step 1208, drying at 30° C. under a vacuum for about 2 hours was conducted.

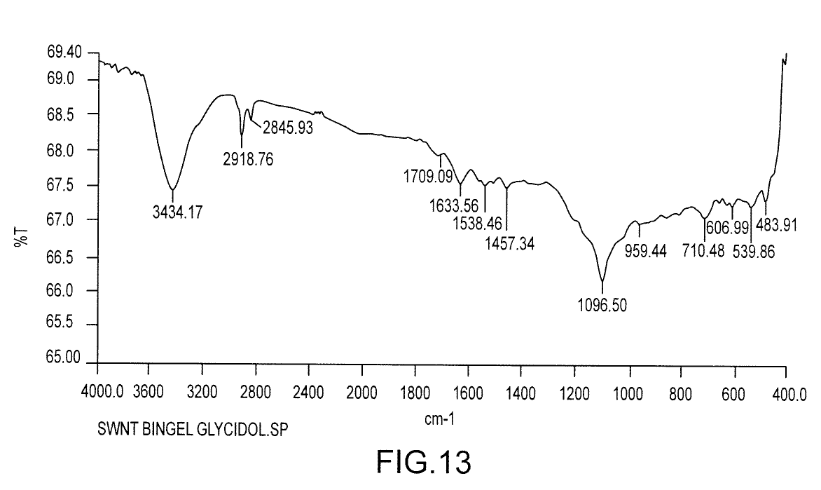

FIG. 13 illustrates Fourier Transform infrared (FTIR) absorption spectra measured on Example 3. The FTIR absorption spectra is reported in Table 3. These results confirm epoxy functionalized SWCNTs as shown at 2918.76 cm-1 CH asymmetric stretching; 2845.93 cm-1 CH symmetric stretching; 1457.34 cm-1 CH2 deformation; and 1096.50 cyclic ethers deformation.

### Strategy D:

In this embodiment, strategy D is directed towards forming epoxy functionalized CNTs. The embodiment includes two steps: step 1, directed towards a cyclopropanation reaction followed by step 2, which is directed towards transesterification reaction. Turning now to Strategy D.

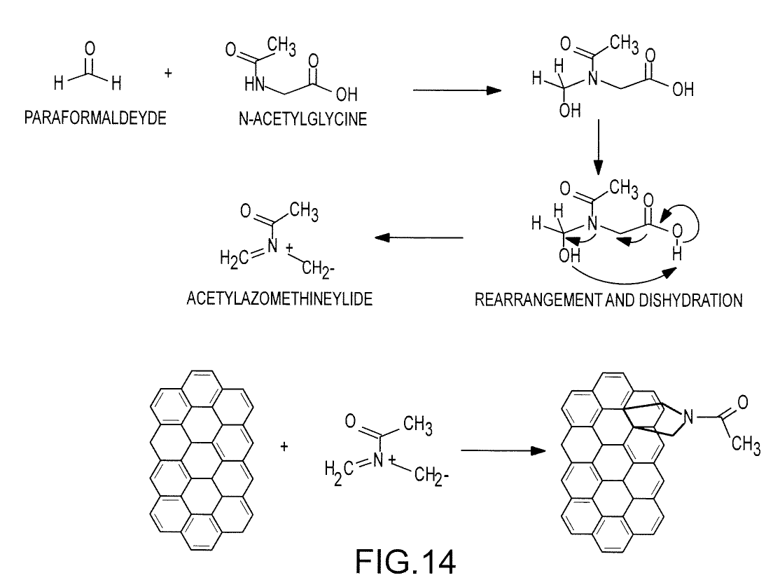

Step 1, sidewall CNTs functionalization through the Prato reaction employing N-acetyl glycine and paraformaldehyde as reactants. Step 2 includes an epoxidation reaction through the Corey-Chaykovsky reaction involving the conversion of a ketonic group in the epoxy ring. The Corey-Chaykovsky reaction is described by Corey, et al., Dimethyloxosulfonium Methylide ((CH)SOCH) and Dimethylsulfonium Methylide ((CH)SCH). Formation and Application to Organic Synthesis, Journal of the American Chemical Society (1965), 87, 6, pp. 1353-1364, which is hereby incorporated by reference. In this embodiment, the chemical strategy is at a design status and the experimental activities are in progress. Step 1 is shown in FIG. 14 illustrating a Prato reaction mechanism with N-acetylglycine and para formaldehyde.

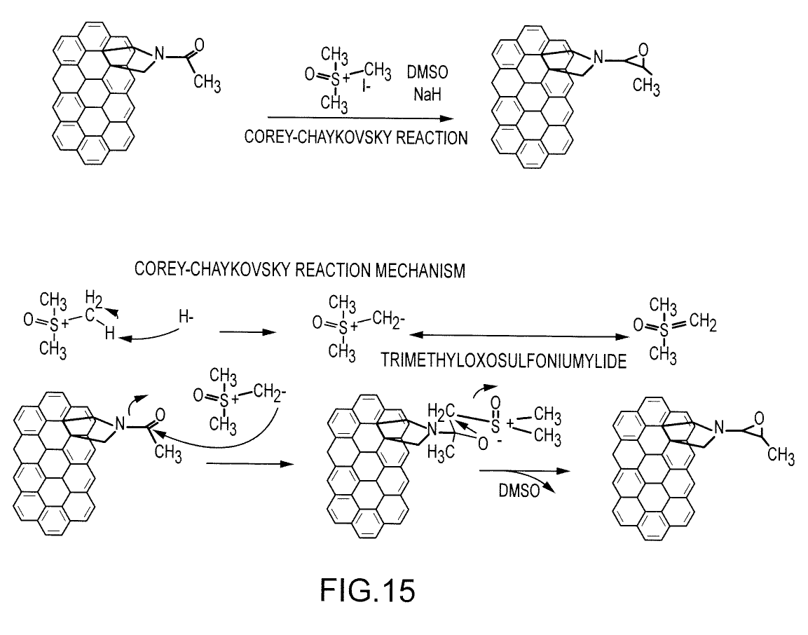

Step 2 is shown in FIG. 15 illustrating a Corey-Chaykovsky epoxydation reaction mechanism. Further, concerning step 2, in order to avoid the use of sodium hydride as strong base, recent literature reports on the Corey-Chaykovsky reaction employing ionic liquids as alternative media in place of dimethylsulfoxide, and potassium hydroxide as base. Accordingly, to avoid the use of sodium hydride, ionic liquids may be implemented; this is described by Chandrasekhar, et al., The first Corey-Chaykovsky epoxidation and cyclopropanation in ionic liquids, Tetrahedron Letters (2003), 44, pp. 3629-3630, which is hereby incorporated by reference as if fully set forth herein. It is believed that the ionic liquids are promising media to create well-stabilized CNTs colloidal solutions reaching a very good level of CNTs dispersion inside as discussed in Park, et al., Covalent Modification of Multiwalled Carbon Nanotubes with Imidazolium-Based Ionic Liquids: Effect of Anions on Solubility, Chem. Mater. (2006), 18, pp. 1546-1551; and Fukushima, et al., Ionic Liquids for Soft Functional Materials with Carbon Nanotubes, Chem. Eur. J. (2007), 13, pp. 5048-5058, all of which are hereby incorporated by reference as if fully set forth herein.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

What is claimed is:

1. A method of forming epoxy functionalized carbon nanotubes (CNTs), comprising the steps of:
functionalizing carbon nanotubes by reacting with organolithium reactants; and
nucleophilic substitution to form the epoxy functionalized carbon nanotubes.

2. The method of claim 1, wherein the functionalizing step comprises reacting the carbon nanotube with a butyllithium solution.

3. The method of claim 2, wherein the butyllithium solution comprises n-butyllithium.

4. The method of claim 1, wherein the carbon nanotube comprises a multiwalled carbon nanotube.

5. The method of claim 1, wherein the nucleophilic substitution step comprises reacting epichlorohydrin with the functionalized carbon nanotubes.

6. A packaging of electronic components, comprising the epoxy functionalized carbon nanotubes made by the method of claim 1.

7. An epoxy functionalized carbon nanotube composite, made by the process of claim 1.

8. A method of forming epoxy functionalized carbon nanotubes (CNTs), comprising the steps of:
functionalizing carbon nanotubes with a Prato reaction; and
nucleophilic substitution to form the epoxy functionalized carbon nanotubes.

9. The method of claim 8, wherein the carbon nanotube comprises a multiwalled carbon nanotube.

10. The method of claim 8, wherein the nucleophilic substitution step comprises reacting epichlorohydrin with the functionalized carbon nanotubes.

11. A packaging of electronic components, comprising the epoxy functionalized carbon nanotubes made by the method of claim 8.

12. An epoxy functionalized carbon nanotube composite, made by the process of claim 8.

13. A method of forming epoxy functionalized carbon nanotubes (CNTs), comprising the steps of:
functionalizing carbon nanotubes with a Bingel reaction; and
transesterification of ethoxylic groups of the functionalized carbon nanotubes by reacting with glycidol molecules to form the epoxy functionalized carbon nanotubes.

14. The method of claim 1, wherein the carbon nanotubes comprise a multiwalled carbon nanotube.

15. A packaging of electronic components, comprising the epoxy functionalized carbon nanotubes made by the method of claim 13.

16. An epoxy functionalized carbon nanotube composite, made by the process of claim 13.

17. A method of forming epoxy functionalized carbon nanotubes (CNTs), comprising the steps of:
functionalizing carbon nanotubes with a Prato reaction; and
epoxidation reaction to form the epoxy functionalized carbon nanotubes.

18. The method of claim 1, wherein the carbon nanotubes comprise a multiwalled carbon nanotube.

19. A packaging of electronic components, comprising the epoxy functionalized carbon nanotubes made by the method of claim 17.

20. An epoxy functionalized carbon nanotube composite, made by the process of claim 17.
